(19) United States
(12) Patent Application Publication
(10) Pub. No.: US 2012/0316408 A1
(43) Pub. Date: Dec. 13, 2012

(54) INDICATORS FOR DETECTING THE PRESENCE OF METABOLIC BY-PRODUCTS FROM MICROORGANISMS

(75) Inventors: Joel R. Gorski, Perrysburg, OH (US); Ram W. Sabnis, Roswell, GA (US)

(73) Assignee: Indicator Systems International, Inc.

(21) Appl. No.: 13/468,863

(22) Filed: May 10, 2012

Related U.S. Application Data
(60) Provisional application No. 61/486,042, filed on May 13, 2011.

(51) Int. Cl. A61B 5/00; A46B 11/00; G01N 21/80; B65D 79/00

(52) U.S. Cl. 600/309; 426/132; 401/143; 435/34

(57) ABSTRACT

pH indicator compositions, solutions and suspensions and pH indicating food storage devices, medical devices and components thereof are provided for visually monitoring, detecting, and/or determining the presence of metabolic by-products from harmful or potentially harmful microorganisms. Also provided are methods of use and preparation of the pH indicator compositions, solutions and suspensions.

### TECHNICAL FIELD

This invention relates generally to indicator compositions, solutions and suspensions useful, for example, in food wraps and medical devices. This invention is also directed to uses of compositions, solutions and suspensions for detecting the presence of bacterial growth as measured by bacterial growth by-products. Such by-products alter the pH of the composition and facilitate a color change as an indication of bacterial contamination.

### BACKGROUND

Detection of incipient bacterial contamination or infection is a long desired goal in preventing human bacterial diseases. In one setting, food products which contain active pathogenic bacteria has been a major source of food borne illness. In another setting, incipient bacterial infection at wound sites is exceptionally difficult to identify. In such cases, the bacterial infection can advance to a stage where treatment protocols are either exceptionally aggressive or not available as in antibiotic resistant bacterial infections.

As to food products intended for consumption, the presence of undesirable bacterial contamination is of significant concern to manufacturers, farmers, packagers, food distributors, wholesalers, retailers, consumers, and to worldwide public health. A particularly worrisome concern is bacterial contamination in packages containing food products for human consumption. The United States boasts of some of the safest food in the world; however, each year approximately one in four individuals suffer from a food borne illness and some 5,000 die from something they have eaten. According to the Center for Disease Control and Prevention, each year in the United States, 76 million people contract some kind of food borne illness, 325,000 are hospitalized and 5,000 fatalities occur due to contamination of consumed food. In Third World countries, it has been estimated that bacterial contaminated food and water kills over two million children each year. Despite those numbers, most food borne infections are undiagnosed and unreported.

Packaging of perishable and edible food products may be susceptible to undesired and undetectable bacterial growth during each stage in the food chain from harvest to consumption. Minimal levels of bacterial contamination (bacterial load) of food is deemed acceptable in food for consumer use. Indeed, regulatory agencies such as the FDA have established limits on bacterial load permitted in the food. Nevertheless, it is very hard to determine if bacterial growth in food alters the bacterial level of the food to unacceptable levels. Food initially safe for consumption may be altered by undetected bacterial growth due to poor handling, improper storage and other factors. At all points in the food chain, it would be of great benefit if there was an unmistakable means to determine that there has been unacceptable bacterial growth occurring on the food.

Still further, bacterial contamination of wounds can lead to serious infection, illness, and even death if the contamination is unnoticed and untreated for even a relatively short period of time. Often times, bacterial infection is first detected by the presence of inflamed red skin around a wound site. Visualization of the wound by skin redness is often at a point where the infection has significantly progressed within the diseased patient.

Examples of such wounds are those generated by use of central venous catheters, cannulae, and related medical devices (hereafter “catheters”) which are inserted and maintained through the skin. As is apparent, catheters are used on a variety of patients, usually in a hospital setting. These catheters provide secure access (e.g., into a patient's blood vessel) and allow for the safe administration of fluids and drugs into the patient or the removal of fluids from the body.

Wounds of all nature carry an inherent risk of bacterial infections. In addition to intentionally created wounds such as those described above, other wounds susceptible to infection include abrasions, burns, surgical incisions, injection sites, and the like.

For example, catheter insertion into the body can cause serious complications. Specifically, catheter related bloodstream infection (CR-BSI) is a serious and potentially life-threatening complication when catheter insertion sites into blood vessel lumen become infected with bacterial microorganisms. Conventional state of the art care now requires that these insertion sites be covered with a wound dressing as a preventive measure against such infections.

A number of factors render such insertion sites especially susceptible to bacterial contamination. Specifically, the catheter essentially compromises the skin's natural protective barrier, providing a direct route to bypass the body's first line of immunity. In addition, upon insertion into the host, the outer surface of the catheter is quickly covered with host proteins that facilitate bacterial attachment and growth. There is also evidence that implanted abiotic material itself causes local attenuation of antimicrobial immune responses, thereby inhibiting a normal immune response against bacterial biofilm formation. Finally, patients who possess the greatest need for catheterization are often immunologically compromised and are therefore more susceptible to bacterial infection.

Catheters themselves are generally infected via one of two general routes, typically by microorganisms that compromise the natural flora surrounding the site of catheter insertion. For example, bacteria may contaminate the catheter along its outer surface, and it is believed that this type of infection often occurs during the initial insertion of the catheter through the skin. Catheters can also be contaminated in their lumenal compartments where fluids flow from contaminated infusate solutions. The most prevalent bacteria found to be the cause of bacterial sepsis are from the exterior flora surrounding the insertion site.

Catheter-related bloodstream infections are notoriously difficult to treat via conventional antibiotic therapy, with associated mortality rates ranging from 12% to 25%. Catheter related bloodstream infection is the most frequent serious complication seen with catheters with infections occurring in as many as 3% to 7% of all catheter placements, which is estimated to affect more than 250,000 patients in U.S. hospitals each year. In addition, these infection complications extend hospital stays, necessitate active intervention on the part of healthcare personnel, and result in driving the estimated annual domestic healthcare cost associated with complications arising from these catheter-related infections to more than nine billion dollars.

The use of a wound covering (sometimes referred to as a “dressing” or “wound dressing”) in conjunction with a catheter is conventional but does not entirely obviate the underlying infection risk as evidenced by the statistics above. Such wound dressings are typically placed proximate the catheter insertion site and contact fluids exuding from that site.

Still further, other wounds such as burns, abrasions, surgical incisions, and the like are particularly susceptible to infection. In hospital settings, infections caused by antibiotic resistant bacteria such as Staphylococcus is a major concern and a cause of morbidity.

Therefore, a need exists for methods and medical devices and wound coverings for the detection of bacterial growth contamination in or about a wound that can readily detect and indicate the presence of microorganisms well before the infection has progressed to the point that it manifests itself by skin redness.

### SUMMARY OF THE INVENTION

In one aspect, this invention relates to an adherent, non-acidic solution or homogeneous suspension which when dry is useful for determining the presence or absence of the growth by-products from contaminating microorganisms said solution or suspension comprises: an adherent biocompatible polymer; a biocompatible liquid; and a plurality of indicator moieties which exhibit a first color or are colorless in the absence of bacterial growth by-products and a second color or are colorless in the presence of bacterial growth by-products.

In another aspect, this invention provides medical devices and food storage devices wherein at least one surface of the device comprises an adherent, non-acidic composition useful for determining the presence or absence of growth by-products from contaminating microorganisms wherein said composition comprises an adherent biocompatible polymer and a plurality of indicator moieties which exhibit a first color or are colorless in the absence of bacterial growth by-products and a second color or are colorless in the presence of bacterial growth by-products. In one embodiment, the polymer is opaque. In another embodiment, the polymer is transparent.

Other aspects of the instant invention relate to methods for detecting the presence of a bacterial infection in a patient having a medical device or component thereof comprising a surface which is implanted or inserted into the patient such that at least a portion of the surface contacts bodily fluids of the patient which method comprises:

a) placing on at least a portion of the surface of the medical device that will be in contact with the bodily fluids of the patient a composition, solution or homogeneous suspension of the invention;

b) detecting the presence or absence of a colorimetric change in the composition or solution or homogeneous suspension; and

c) correlating the presence or absence of a colorimetric change in the composition, solution or suspension to the presence or absence of an active bacterial infection in the patient wherein a colorimetric change correlates to the presence of an active bacterial infection and the lack of a colorimetric change correlates to the absence of an active bacterial infection.

In another of its method aspects there is provided a method for detecting the presence of bacterial contamination in food contained in a food storage device comprising a surface such that at least a portion of the surface contacts the food which method comprises:

a) placing on at least a portion of the surface of the food storage device that will be in contact with the food a composition, solution or homogeneous suspension of the invention;

b) detecting the presence or absence of a colorimetric change in the composition, solution or homogeneous suspension; and

c) correlating the presence or absence of a colorimetric change in the composition, solution or homogeneous suspension to the presence of an active bacterial contamination in the food wherein a colorimetric change correlates to the presence of an active bacterial infection and the lack of a colorimetric change correlates to the absence of an active bacterial infection.

This invention also provides for diagnostic kits for use in determining the absence or presence of bacterial infection. In one embodiment, the kit comprises a device which, at its distal end, comprises a composition comprising the adherent polymer and the pH indicators and, at its proximal end, a handling means, or a portion dedicated to be handled, and a shaft between the distal and proximal end.

In one aspect, there is a method for determining the presence of bacterial infection in a fluid composition which method comprises contacting said distal end of device with fluid for sufficient time to determine a color change.

### DETAILED DESCRIPTION OF THE INVENTION

Throughout this application, the text refers to various embodiments relating to compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the instant invention.

Definitions

As used herein, certain terms may have the following defined meanings. As used in the specification and claims, the singular form “a,” “an” and “the” include singular and plural references unless the context clearly dictates otherwise.

The term “comprising” is intended to mean that the compounds and methods include the recited elements, but not excluding others. “Consisting essentially of” when used to define compositions and methods, shall mean excluding other elements of any essential significance to the compounds or methods. “Consisting of” shall mean excluding more than trace elements of other ingredients for claimed compounds and substantial method steps. Embodiments defined by each of these transitional terms are within the scope of this invention. Accordingly, it is intended that the processes and compositions can include additional steps and components (comprising) or alternatively include additional steps and compounds of no significance (consisting essentially of) or alternatively, intending only the stated methods steps or compounds (consisting of).

Neutral pH has a value of 7.0. As used herein, the term “neutral pH” also includes low acid pH of from about 6 to below 7 and low basic pH of from above 7 to up to about 8.

The term “acidic” as used herein refers to an acidic pH range generally produced from by-products of bacterial growth. Such acidic pHs generally range from above 1 to about 5 and, preferably, a pH range of from 2 to about 5. A strong acid has a pH of below 2.0.

The term “transparent” refers to a polymer or liquid which is sufficiently transparent to visible light that a viewer can readily see through the layer.

The term “non-transparent” refers to a composition, solution, homogeneous suspension, or layer which is opaque.

The term “threshold level of bacterial by-products” refers to the amount of by-products produced by bacteria such that the pH changes sufficiently to effect a change in the color of the indicator from a first color in the absence of a threshold level of bacterial by-products to a second color in the presence of a threshold level of bacterial by-products. Preferably, the threshold level of bacterial by-products is a level at or below the level produced by a minimum amount of bacteria growth that would cause concern when present on food or at a medical device insertion site or point of contact with bodily fluids. As used herein, the term “active bacterial infection” refers to an infection that produces a level of bacterial by-products above the threshold level of bacterial by-products.

The term “indicator” refers to a substance capable of changing color with a change in pH caused when a threshold amount of bacterial by-products are produced. In one embodiment, the indicator is a pH indicator. Such pH indicators are sometimes referred to herein as “pH indicating moieties.” Bacterial by-products include, but are not limited to, gaseous carbon dioxide, hydrogen sulfide, sulfur dioxide, hydrogen, ammonium, lactate, and mixtures thereof. Mixtures of these by-products with moisture result in the formation of acids such as carbonic acid, sulfuric acid, ammonium hydroxide, lactic acid, or mixtures thereof. When a sufficient amount of acid is generated, the indicator produces a color change that is readily discernable by even an untrained observer.

Examples of pH indicators include xylenol blue (p-xylenolsulfonephthalein), bromocresol purple (5′,5″-dibromo-o-cresolsulfonephthalein), bromocresol green (tetrabromo-m-cresolsulfonephthalein), cresol red (o-cresolsulfonephthalein), m-cresol purple, thymol blue, o-cresolphthalein, thymolphthalein, crystal violet, malachite green, phenolphthalein, phenol red, bromothymol blue (3′,3″-dibromothymolsulfonephthalein), p-naphtholbenzein (4-[alpha-(4-hydroxy-1-naphthyl)benzylidene]-1(4H)-naphthalenone), neutral red (3-amino-7-dimethylamino-2-methylphenazine chloride), pentamethoxy red, hexamethoxy red and heptamethoxy red, and combinations thereof. In one embodiment, the pH indicators are hexamethoxy red and/or heptamethoxy red or derivatives thereof.

The term “biocompatible polymer” refers to polymers which, in the amounts employed, are non-toxic, chemically inert, and substantially non-immunogenic when used internally in the patient and which are substantially insoluble in blood.

The term “bacteria” as used herein refers to any bacteria that may be present in either food, a wound site, or a medical device regardless of origin and that may further be a potential health hazard. Examples of bacteria detectable by the pH indicator composition, solution or suspension provided herein include Staphylococcus aureus, Staphylococcus epidermidis, Streptococcus mitis, Streptococcus sanguis, Enterococcus faecium, Escherichia coli, Enterobacter cloacae, Enterobacter aerogenes, Enterococcus faecalis, Pseudomonas aeruginosa, Klebsiella pneumonia, Candida albicans, Bacillus, Brucella, Campylobacter, Clostridium, Escherichia coli, Listeria monocytogenes, Salmonella, Streptococcus, Pseudomonas aeruginosa, Staphylococcus aureus, Shigella, Vibrio, Yersinia, gram negative bacilli, or a combination thereof.

The term “catheter” includes any and all known catheters which puncture the skin and are used for delivering fluids, medicaments, etc. into the body, assisting in the elimination of fluids from the body, and/or for diagnostic purposes. Such catheters include central venous catheters, diagnostic catheters, drainage catheters, and the like. Also included within the term “catheter” are cannulae which are conventional, well known, tubes inserted into the body by puncture through the skin, for the delivery or removal of fluids. Cannulae normally come with a trocar which permits puncturing of the body.

The term “bacterial contamination” refers to the growth of microorganisms, such as bacteria, on food. As used herein, rancidity, which is a breakdown of the cellular matrix of the tissue or meat via protein denaturization process and release of proteins (enzymes) to the extracellular spaces of the tissue, is not detected by the invention.

The term “bodily fluids” refers to fluids that are derived from the body, fluids that are intended to be administered into the body, and fluids that contact the body.

The term “rheological modifier” as used herein refers to a component which when added to the solution or homogeneous suspension imparts high rest viscosity or yield stress of the composition but permits the solution or homogeneous suspension to readily flow under shear stress.

“Surfactants” are those substances which enhance flow and/or aid dispersion by reducing surface tension when dissolved in aqueous solutions, or that reduce interfacial tension between two liquids, or between a liquid and a solid. Surfactants also impede the interaction between the rheological modifier and other components of the system. This allows a more fully developed rheological modified system.

The terms “micronize” and “micronized” generally refer to a process, or particles which have been processed, such that their diameters/sizes are within the general range of microparticles and/or nanoparticles.

Methods and Compositions

In one aspect, this invention provides an adherent, non-acidic solution or homogeneous suspension which when dry is useful for determining the presence or absence of the growth by-products from contaminating microorganisms said solution or suspension comprises an adherent biocompatible polymer; a biocompatible liquid; and a plurality of indicator moieties which exhibit a first color or are colorless in the absence of bacterial growth by-products and a second color or are colorless in the presence of bacterial growth by-products.

The solution or homogeneous suspension is useful as a coating on food storage or medical devices or components thereof for detecting the presence or absence of bacterial contamination. In certain embodiments, the biocompatible polymer is a non-acidic and/or non-transparent polymer. In other embodiments, the polymer is a transparent polymer. Non-limiting examples of polymers useful in the instant invention include methylcellulose, hydroxypropyl ethylcellulose, hydroxyethyl cellulose, ethyl cellulose, hydroxypropyl methylcellulose, polyvinyl alcohol, polyvinyl chloride, polyvinylidene chloride, polyhydroxyethyl methacrylate, polyhydroxypropyl methacrylate, polyglycerol methacrylate, copolymers of hydroxyethyl methacrylate, hydroxypropyl methacrylate, glycerol methacrylate, methacrylic acid, aminoacrylate, aminomethacrylate, polyvinylpyridine, polyamides, hydroxypropyl cellulose, ethylhydroxyethylcellulose, carboxymethyl cellulose, cellulose acetate, cellulose acetate butyrate, cellulose acetate propionate, cellulose nitrate, polyvinyl acetate, polyvinyl alcohol, copolymers of polyvinylacetate and polyvinyl alcohol, hydroxy-modified copolymers of vinyl acetate and vinylchloride, polyesters and polyurethanes containing at least 10% by weight of polyethylene oxide, styrene/methacrylic acid/hydroxyethyl methacrylate copolymers, styrene/methacrylic acid/hydroxypropyl methacrylate copolymers, methylmethacrylate/methacrylic acid copolymers, ethyl methacrylate/styrene/methacrylic acid copolymers, ethyl methacrylate/methyl methacrylate/styrene/methacrylic acid copolymers, polytetrafluoroethylene and hydrophilic cellulose copolymers, a host of other polymers, said polymer or mixture thereof. Selection of the polymer is dictated by the proposed application or use. In certain embodiments, the polymer is ethyl cellulose.

Examples of biocompatible liquids suitable for use in this invention include ethylene dichloride, methanol, ethanol, or ethyl lactate. In one embodiment, the liquid is selected from the group consisting of ethylene dichloride, methanol, ethanol, or ethyl lactate. In another embodiment, the liquid is ethanol. In yet further embodiments, the liquid is non-acidic or transparent.

In one embodiment the composition comprises a 5% (w/v) solution of a biocompatible polymer in a biocompatible liquid. In one embodiment the biocompatible polymer is ethyl cellulose. In another embodiment, the composition comprises a 1% (w/v) solution of a biocompatible polymer in a liquid, or, alternatively, the w/v solution of the biocompatible polymer in a liquid is 2%, 3%, 4%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%.

Solutions and suspensions also optionally include materials that modify certain properties of the solution or suspension to make such compositions more easily applied to substrates or more easily dispensed from applicators. These materials include, for example, surfactants, rheological modifiers (thixotropic agents), and titanium dioxide. Other components are well known within the art.

In one embodiment, there is provided a non-acidic solution or homogeneous suspension which when dried is useful for determining the presence or absence of the growth by-products from contaminating microorganisms said solution or suspension comprises: ethyl cellulose; ethanol; and a plurality of indicator moieties which exhibit one color or are colorless in the absence of bacterial growth by-products and change color in the presence of bacterial growth by-products.

In another embodiment, there is provided an adherent composition useful for determining the presence or absence of the growth by-products from contaminating microorganisms when said composition is adhered to a surface of a substrate and further wherein said composition comprises: an adherent biocompatible polymer; and a plurality of indicator moieties which exhibit a first color in the absence of bacterial growth by-products and a second color in the presence of bacterial growth by-products. In a related embodiment there is provided a substrate comprising on at least one surface thereof the composition according to this invention.

The composition can be dried to allow for the removal of the solvent. Methods of drying include the application of hot air, indirect or contact drying as in drum drying or vacuum drying, freeze drying, natural air drying, and centrifugation.

In certain embodiments the indicator moieties are pH indicator moieties uniformly dispersed throughout the composition, solution, or homogeneous suspension. The pH indicator moieties allow for the visual detection of bacterial growth. In certain embodiments the pH indicating moieties are selected from heptamethoxy red and hexamethoxy red or a combination thereof. In another embodiment, the pH indicating moieties are a derivative of heptamethoxy red or hexamethoxy red. Other examples of pH indicator moieties useful in the invention include xylenol blue (p-xylenolsulfonephthalein), bromocresol purple (5′,5″-dibromo-o-cresolsulfonephthalein), bromocresol green (tetrabromo-m-cresolsulfonephthalein), o-cresol red (o-cresolsulfonephthalein), m-cresol purple, thymol blue, o-cresolphthalein, thymolphthalein, crystal violet, malachite green, phenolphthalein, phenol red, bromothymol blue (3′,3″-dibromothymolsulfonephthalein), p-naphtholbenzein (4-[alpha-(4-hydroxy-1-naphthyl)benzylidene]-1(4H)-naphthalenone), neutral red (3-amino-7-dimethylamino-2-methylphenazine chloride), pentamethoxy red, hexamethoxy red and heptamethoxy red, and combinations thereof.

The pH indicating moieties in the composition, solution or suspension are employed in an amount effective for detecting a color change thereby evidencing a change in pH. As used herein, the term “detection” denotes a color-change either visible by human eye having ordinary vision. Alternatively, instrumentation may be used to detect the color change. In some embodiments the pH indicating moiety is employed in an amount of about 0.01% w/w to about 10% w/w relative to the weight of the composition, solution or suspension. In some embodiments the pH indicating moiety is employed in an amount of about 1% w/w to about 3% w/w.

In a preferred embodiment the mass ratio of the pH indicator, the biocompatible polymer, and the biocompatible liquid ranges from 0.01:1:79 to about 0.25:1:1.2 of pH indicator: biocompatible polymer:biocompatible liquid by mass. Particularly preferred ratios include 1:5:76; 3:5:76; 3:3:78; 1:3:78; 5:5:76; 1:7:75; 3:7:75; 5:7:75, 1:9:73; 3:9:73; 5:9:73; and 7:9:73 by mass.

In another embodiment the mass ratio of pH indicator to the biocompatible polymer ranges from about 0.00025 to about 10. Preferred rations include 0.1, 0.2, 0.25, 0.33, 0.4, 0.5, 1, 1.7, and 5 by mass.

In some embodiments, the composition, solution or suspension comprises a sufficient amount of pH indicator moieties to provide visible color change in at least a portion of the composition, solution, or homogeneous suspension upon contact with bacterial growth by-products.

The pH indicating moieties detect pH change associated with by-products of bacterial growth. These by-products include, among others, gaseous carbon dioxide, hydrogen sulfide, sulfur dioxide, hydrogen, ammonium, lactate, and mixtures thereof. Mixtures of the by-product with moisture result in the formation of acids such as carbonic acid, sulfuric acid, ammonium hydroxide, lactic acid, or mixtures thereof that react with the indicator to produce a color change. The term “by-products” with reference to bacteria, refer to the gases that are expelled from the bacteria due to their natural growth of populations in numbers. Such gases can be in the vapor state or can combine with water or be hydrolyzed to form an acid such as sulfuric acid, carbonic acid, hydrogen sulfide or other gaseous or water vapor state which lowers the pH of the immediate environment with increasing concentrations of the gas vapor or water vapor combination.

In some embodiments, the acid is generated from a bacteria or is formed by reaction of a bacterial by-product with water, said bacterial by-product is selected from the group consisting of carbon dioxide and sulfur dioxide.

It is contemplated that in addition to bacteria, microbes detectable by the packaging materials include, among others, viral and fungal microbes. Among bacteria whose growth in food, bodily fluids, and medical devices or components thereof can be detected by the methods and compositions described herein include but are not limited to Staphylococcus aureus, Staphylococcus epidermidis, Streptococcus mitis, Streptococcus sanguis, Enterococcus faecium, Escherichia coli, Enterobacter cloacae, Enterobacter aerogenes, Enterococcus faecalis, Pseudomonas aeruginosa, Klebsiella pneumonia, Candida albicans, Bacillus, Brucella, Campylobacter, Clostridium, Escherichia coli, Listeria monocytogenes, Salmonella, Streptococcus, Pseudomonas, Staphylococcus, Shigella spp., Vibrio spp., Yersinia spp., gram negative bacilli, coliform or spore forming bacteria and other food borne or air borne pathogens or a mixture of such microbes known to be involved in food contamination or the contamination of medical devices or components thereof. Particular strains have been identified as associated with fresh vegetables. For example, Escheria coli O157:H7 was associated with prepackaged spinach: “Investigation of an Escheria coli O157:H7 Outbreak Associated with Dole Pre-Packaged Spinach,” California Food Emergency Response Team Final Report, Mar. 21, 2007 (available from the California Department of Health Services, Food and Drug Branch, P.O. Box 997435, MS 7602, Sacramento, Calif. 95899-7435 and also available from U.S. Food and Drug Administration San Francisco District, 1431 Harbor Bay Parkway, Alameda, Calif. 94502.)

The compositions of the invention can be a solution or a suspension. In one embodiment the composition is micronized or sprayable. Techniques to micronize solutions or suspensions are well known in the art. Traditional micronization techniques are based on friction to reduce particle size. Such methods include milling, bashing and grinding. A typical industrial mill is composed of a cylindrical metallic drum that usually contains steel spheres. As the drum rotates the spheres inside collide with the particles of the solid, thus crushing them towards smaller diameters. In the case of grinding, the solid particles are formed when the grinding units of the device rub against each other while particles of the solid are trapped in between.

Methods like crushing and cutting are also used for reducing particle diameter, but produce more rough particles compared to the two previous techniques (and are therefore the early stages of the micronization process). Crushing employs hammer-like tools to break the solid into smaller particles by means of impact. Cutting uses sharp blades to cut the rough solid pieces into smaller ones.

Modern methods use supercritical fluids in the micronization process. The most widely applied techniques of this category include the RESS process (Rapid Expansion of Supercritical Solutions), the SAS method (Supercritical Anti-Solvent) and the PGSS method (Particles from Gas Saturated Solutions).

In the case of RESS, the supercritical fluid is used to dissolve the solid material under high pressure and temperature, thus forming a homogeneous supercritical phase. Thereafter, the solution is expanded through a nozzle and small particles are formed. At the rapid expansion point right at the opening of the nozzle there is a sudden pressure drop that forces the dissolved material (the solid) to precipitate out of the solution. The crystals that are instantly formed enclose a small amount of the solvent that, due to the expansion, changes from supercritical fluid to its normal state (usually gas), thus breaking the crystal from inside-out. At the same time, further reduction of size is achieved while the forming and breaking crystals collide with each other at the vicinity of the nozzle. The particles that are formed this way have a diameter of a few hundreds of nanometers.

In the SAS method, the solid material is dissolved in an organic solvent and a supercritical fluid is then also forced by means of pressure to dissolve in the system. In this way, the volume of the system is expanded, thus lowering the density, and therefore also the solubility of the material of interest is decreased. As a result, the material precipitates out of the solution as a solid with a very small particle diameter.

In the PGSS method the solid material is melted and the supercritical fluid is dissolved in it, like in the case of the SAS method. However, in this case the solution is forced to expand through a nozzle, and in this way nanoparticles are formed.

In all three methods described, the effect that causes the small diameter of the solid particles is the supersaturation that occurs at the time of the particle formation, like it was described in more detail in the case of the RESS process. The PGSS method has the advantage that because of the supercritical fluid, the melting point of the solid material is reduced. Therefore, the solid melts at a lower temperature than the normal melting temperature at ambient pressure. In addition, all these new techniques do not demand long processing times, like in the case of the traditional methods. As a result, they are thought to be more appropriate when thermo-labile materials need to be processed (like pharmaceuticals and foodstuff ingredients).

The solutions or suspensions are useful for coating medical devices or food storage devices to aid in the detection of bacterial contamination of such devices. In a related embodiment, there is provided an applicator for dispensing the solution or homogeneous suspension comprising a storage chamber that holds the solution or homogeneous suspension and a dispensing mechanism. Non-limiting examples of dispensing mechanism include a sprayer, a brush, a rotogravure or other type of printing or coating press, or an elongated shaft with a dispensing tip. The tip of the elongated shaft can be a brush, a non-flexible material, a flexible polymeric material, or a sponge.

The viscosity of the composition may vary depending on the type of applicator used. In some embodiments the viscosity of the composition is from at least about 1 cP to about 20,000 cP, or from about 500 cP to about 1,500 cP, or from about 1 cP to about 500 cP, or from about 1 cP to about 2,000 cP, or from about 100 cP to about 1,000 cP, or from about 1 cP to about 50 cP or from about 1,000 cP to about 1,500 cP, or from about 500 cP to about 1,000 cP, or from about 1 cP to about 100 cP or from about 100 cP to about 200 cP.

When used in a composition for spraying on a substrate, the viscosity is from about 1 cP to about 500 cP, or alternatively, from about 1 cP to about 1000 cP, or from about 1 cP to about 100 cP, or from about 100 cP to about 200 cP, or from about 200 cP to about 300 cP, or from about 400 cP to about 500 cP, or from about 10 cP to about 50 cP.

In some embodiments the solution or homogeneous suspension or the composition further comprises other components that modify the certain properties of the solution such as the viscosity, solubility, dry time, surface tension, cross-linking, surface hardness, opacifiers, and the like. Components that modify such properties of compositions include, for example, rheological modifiers, surfactants, solvents, cross linkers, and surface stabilizers. Some non-limiting examples of rheological modifiers include fumed silica and hydroxyl containing modifiers. Hydroxyl-containing rheological modifiers include by way of example only, polymers such as poly(acrylates) such as poly(2-hydroxyethylacrylat- es), poly(alkenes) such as copolymers of ethylene and maleic acid, polyvinylalcohol, oxidized poly(alkenes), cellulosic polymers and copolymers [including hydroxypropylcellulose, hydroxypropylmethylcellulose, carboxymethylcellulose, sodium hydroxyethylcellulose, hydroxyethylcellulose and methylcellulose], poly(methacrylates) such as poly(2-hydroxyethylmethacrylates), poly(saccharides), poly(siloxanes), carrageenan, guar, xanthan gum, locus bean gum, homo- and co-polymers of mannuronic acid and glucuronic acid, and the like.

Surfactants may be anionic, cationic, and nonionic. Surfactants include detergents, wetting agents, and emulsifiers. Suitable cationic surfactants include organic amines and organic ammonium chlorides (e.g., N-tallow trimethylene diamine diolealate and N-alkyl trimethyl ammonium chloride) and the like. Suitable anionic surfactants include, by way of example sulfosuccinates, carboxylic acids, alkyl sulfonates, octoates, oleates, stearates, and the like. Suitable nonionic surfactants, include by way of example, bridging molecules discussed above, Tritons, Tweens, Spans and the like. Polyfunctional additives such as glycerin and various glycols may be added. The adjustment of pH by the addition of potassium or sodium hydroxide ionizes silanols and alters the composition's rheology.

Exemplary surface stabilizers include, but are not limited to, known organic and inorganic excipients, as well as peptides and proteins. Such excipients include various polymers, low molecular weight oligomers, natural products, and surfactants. Useful surface stabilizers include nonionic surface stabilizers, anionic surface stabilizers, cationic surface stabilizers, and zwitterionic surface stabilizers. Combinations of more than one surface stabilizer can be used in the invention. Representative examples of surface stabilizers include, but are not limited to, foregoing alone or in combination: hydroxypropyl methylcellulose (HPMC); dioctyl sodium sulfosuccinate (DOSS); sodium lauryl sulfate (SLS) a.k.a. sodium dodecyl sulfate (SDS); hydroxypropyl cellulose grade HPC-SL (viscosity of 2.0 to 2.9 mPas, aqueous 2% W/V solution, 20 DEG C, Nippon Soda Co., Ltd.); polyvinylpyrrolidone (PVP) such as Kollidone® K12 sold by BASF a.k.a. Plasdone® C-12 sold by ISP Technologies, Inc. (USA), Kollidone® K17 sold by BASF a.k.a. Plasdone® C-17 sold by ISP Technologies, Inc. (USA), Kollidone® K29/32 sold by BASF a.k.a. Plasdone® C-29/32 sold by ISP Technologies, Inc. (USA); sodium deoxycholate; block copolymers based on ethylene oxide and propylene oxide commonly known as poloxamers which are sold under the Pluronic® name by BASF (sold under the trade name Lutrol® in EU) and include Pluronic® F 68 a.k.a. poloxamer 188, Pluronic® F 108, a.k.a. poloxamer 338, Pluronic® F 127 a.k.a poloxamer 407; benzalkonium chloride a.k.a. alkyldimethylbenzylammonium chloride; copolymers of vinylpyrrolidone and vinyl acetate commonly known as copovidone sold under the tradename Plasdone® S-630 by ISP Technologies, Inc. (USA); lecithin; polyoxyethylene sorbitan fatty acid esters commonly known as polyoxyethylene 20 sorbitan monolaurate a.k.a. “polysorbate 20”, polyoxyethylene 20 sorbitan monopalmitate a.k.a. “polysorbate 40,” polyoxyethylene 20 sorbitan monooleate a.k.a. “polysorbate 80” sold under the trade names Tween® 20, Tween® 40 and Tween® 80, respectively, by ICI Americas; albumin; lysozyme; gelatin; macrogol 15 hydroxystearate sold as Solutol® 15 by BASF; tyloxapol, and polyethoxylated castor oils sold under the trade name Cremophor® EL by BASF. Additional examples of useful surface stabilizers include, but are not limited to, polymers, biopolymers, polysaccharides, cellulosics, alginates, phospholipids, poly-n-methylpyridinium chloride, anthryul pyridinium chloride, cationic phospholipids, chitosan, polylysine, polyvinylimidazole, polybrene, polymethylmethacrylate trimethylammonium bromide (PMMTMABr), hexyldecyltrimethylammonium bromide (HDMAB), and polyvinylpyrrolidone-2-dimethylaminoethyl methacrylate dimethyl sulfate.

The compositions, solutions or homogeneous suspensions of the instant invention can be used for the detection of bacterial contamination in food or on medical devices or components of medical devices. When the composition, solution or suspension is used in a medical application, the biocompatible polymer and the biocompatible solvent are components that are medical grade safe components according to the Food and Drug Administration. In one example the components meet the requirements for long-term skin contact as established by the United States Food and Drug Administration. When the compositions, solutions or suspensions are used as food storage devices or used to detect contamination from food, the biocompatible polymer and the biocompatible solvent are food grade safe components according to the Food and Drug Administration.

Certain aspects of this invention relate to the detection of bacterial contamination on the surface of the body. The skin is the largest organ of the human body. One of the key functions that the skin performs is to protect the body's “insides” from the external environment by acting as a barrier and/or a filter between the “outside” and the “inside” of the body. The skin has other functions such as regulating the body's temperature and allowing for the excretion of some selected body wastes and toxins.

The acid/base balance is very important to metabolic health and plays a very important role in human physiology. The measure of acids and bases is conducted by determining the pH level, which is the inverse log of the hydrogen ion concentration. The pH scale or range is between 0 and 14 with 7 being neutral. Acids range between pH 0 to less than pH 7 and bases from above pH 7 to pH 14. pH 7 is defined as neutral—neither acidic nor basic. Weak acids are between pH 5.5 and less than pH 7 and weak bases between above pH 7 and pH 8.5.

Provided are substrates comprising on at least one surface thereof a pH indicating composition, solution or suspension of this invention. In some embodiments, the biocompatible liquid is removed from the composition, solution or suspension. Such removal can be performed by aforementioned drying techniques such that the biocompatible liquid content is less than 10% by weight, or, alternatively, less than 9%, or less than 8%, or less than 7%, or less than 6%, or less than 5%, or less than 4%, or less than 3%, or less than 2%, or less than 1%, or less than 0.5%, or less than 0.1%, by weight. In certain embodiments, the substrate is human skin. In other embodiments the substrate is a medical device or component thereof. Preferably, the surface comprising said composition, solution or suspension is one which contacts bodily fluids.

In one embodiment, the medical device is a wound dressing having coated on the surface that contacts the skin or wound of the patient the pH indicator composition, solution or suspension according to this invention. In another embodiment, the medical device is a catheter, a catheter sheath, a wound dressing, a surgical drape, surgical tape, or an implant. In yet further embodiments, there is provided a component of a medical device that contains the pH indicator composition, solution or suspension. One example of a component of a medical device is one in which the bodily fluids are pumped away from the wound and collected in a storage tank. The indicator would be present only in the storage tank. Similarly, a sheath over a catheter tip would be a component of a catheter. In some embodiments, the component is in direct contact with the patient such as a covering over a catheter or an intravenous insertion site. In other embodiments, the component is not in contact with the patient but is in contact with bodily fluids of the patient.

Wound sites susceptible to contamination by microorganisms include skin wounds, abrasions, burns, openings, surgical incision sites, puncture sites, and catheter insertion sites containing, for example, central venous catheter or other catheters used for insertion into the lumen of an artery or vein.

In further embodiments, the substrate is a food or liquid storage device. Preferably, the surface comprising said composition, solution or suspension is one which contacts food or liquid. The food or liquid storage device can be any device capable of storing food. Examples of such devices include bottles, jugs, food wraps, food containers, bulk containers, barrels, crates, bushel baskets, sacks, and bulk bags.

In another embodiment, the substrate is a portable tag that can be placed in close proximity to food, such as in a food container comprising food, so as to detect bacterial contamination in the food. In a related embodiment, the composition, solution or suspension is coated over a portion of the portable tag in the form of a warning (e.g., DO NOT EAT or CAUTION) which would become visible with a change in pH due to bacterial contamination.

In certain embodiments the food or storage device or portable tag has attached thereto or printed thereon a machine recognizable code. The code can be useful for tracking the origin of contaminated food products. In a related embodiment, the code is a barcode. In another embodiment, the code is an RFID tag.

Food refers to any edible substance including solids and liquids such as meats, fish, vegetables, milk, milk products such as yogurt, cottage cheese, ice cream, etc., fruit and the like. Preferably, the food used in combination with the pH indicator composition, solution or suspension of this invention are those which, when contaminated by microbes, provide for a detectable byproduct either from the food or the microbe that alters the pH of the food in a detectable manner.

In some aspects of these embodiments, the composition, solution or suspension is coated over a portion of a food storage device in the form of a warning (e.g., DO NOT EAT or CAUTION) which would become visible with a change in pH due to bacterial contamination.

Another aspect of the instant invention relates to a food probe for determining the presence or absence of the growth by-products from contaminating microorganisms comprising a surface such that at least a portion of the surface contacts the food which probe contains on a portion of the surface which contacts said food or liquid a composition, solution or suspension according to this invention.

Some embodiments of the invention relate to the detection of by-products of contaminating bacterial growth in a packaged food product to provide an early warning of possible microbial growth occurring during storage in that package. These food products may be within the group commonly known as the low acid foods comprising meats, poultry, dairy, seafood and the like. These low acid foods have an inherent pH of near neutral or pH 7 or between pH 7.4 and 6.2. Foods known to be within the class referred to as medium acid foods are soups and pasta and have an inherent pH of 4.5 to 5.0. Foods that are known to be within the class referred to as acid foods are fruits and vegetables with an inherent pH between 3.7 and 4.5. Food known to be within the class referred to as high acid foods include lemons and pickled products with an inherent pH of between 2.3 and 3.7. In certain embodiments, food products other than those within the low acid range that have a more acidic characteristic may not be included in the applicable food product packaging for use with certain embodiments of this invention when the inherent lower pH values of the foods cause a reaction with the pH indicator of the packaging material and signal a false-positive result.

As pentamethoxy red, hexamethoxy red and heptamethoxy red have significantly different pKa's (they change colors at different pHs), it is within the skill of the art to select the appropriate indicator relative to the acidity of the food stored within the pH indicating food storage device of this invention.

The pH indicator composition, solution or suspension of the invention can be applied to medical devices or food storage devices through use of an applicator. Alternatively, the composition, solution or suspension can be applied using any number of different coating technologies, including: flexographic coating, air knife coating, spray technologies, curtain coating, gap coating (knife over roll, knife over blanket, floating knife, etc.), gravure coating, immersion (dip) coating, mayer bar (meyer bar, metering rod), reverse roll coating (L-head, nip-fed, pan-fed), silk screen, rotary screen, or slot die (slot, extrusion). The choice of which coating technology might be selected is determined, in part, by the desired characteristics of the resulting composition, solution or suspension (i.e., thickness). The coating solution can be modified in various ways to affect viscosity, dry speed, foaming, shell hardness, surface tension, production costs, etc.

Methods of the invention relate to detecting bacterial contamination in food products, medical devices, or components thereof. Accordingly, in one of its method aspects, there is provided a method for detecting the presence of a bacterial infection in a patient having a medical device or component thereof comprising a surface which is implanted or inserted into the patient such that at least a portion of the surface contacts bodily fluids of the patient which method comprises:

a) placing on at least a portion of the surface of the medical device that will be in contact with the bodily fluids of the patient the composition, solution or homogeneous suspension according to this invention;

b) detecting the presence or absence of a colorimetric change in the composition, solution or suspension; and

c) correlating the presence or absence of a colorimetric change in the composition, solution or suspension to the presence of an active bacterial infection in the patient wherein a colorimetric change correlates to the presence of an active bacterial infection and the lack of a colorimetric change correlates to the absence of an active bacterial infection.

In another of its method aspects there is provided a method for detecting the presence of bacterial contamination in food contained in a food storage device comprising a surface such that at least a portion of the surface contacts the food which method comprises:

a) placing on at least a portion of the surface of the food storage device that will be in contact with the food a composition, solution or homogeneous suspension according to this invention;

b) detecting the presence or absence of a colorimetric change in the composition, solution or homogeneous suspension; and

c) correlating the presence or absence of a colorimetric change in the composition, solution or homogeneous suspension to the presence of an active bacterial contamination in the food wherein a colorimetric change correlates to the presence of an active bacterial infection and the lack of a colorimetric change correlates to the absence of an active bacterial infection.

### EXAMPLES

In the examples below as well as throughout the application, the following abbreviations have the following meanings If not defined, the terms have their generally accepted meanings.

° C.=degrees Celsius

DE=Diatomaceous earth

° F.=Degrees Fahrenheit

g=Gram

IPA=Isopropyl Alcohol

kg=Kilogram

L=Liter

M=Molar

° C.=Degrees Celsius

mbar=Millibar

mg=Milligram

min=Minutes

mL=Milliliter

MW=Molecular Weight

m/z=Mass/Charge

PE=Polyethylene

PVOH=Polyvinyl Alcohol

RT=Room Temperature

w/w=Weight to weight

### Example 1

### Preparation of Heptamethoxy Red in Gram Scale

### Step 1: Synthesis of Methyl 2,4,6-trimethoxybenzoate (CAS #29723-28-2)

2,4,6-trimethoxybenzoic acid (CAS #570-02-5) (5.61 g, 26.42 mmol) was suspended in 20 mL of methanol (CAS #67-56-1). Concentrated sulfuric acid (CAS #7664-93-9) (1 mL) was added to the mixture, and the reaction heated to reflux for 24 hrs. The reaction was cooled to room temperature, and the methanol (CAS #67-56-1) removed in vacuo. The residues were taken up in 50 mL 5% NaHCO₃ (CAS #144-55-8) and extracted with hexane (CAS #110-54-3) until all the solids had dissolved. The hexane extract was dried over anhydrous Na₂SO₄ (CAS #7757-82-6), filtered, and rotovapped to dryness to give the desired product, methyl 2,4,6-trimethoxybenzoate (CAS #29723-28-2), as a white crystalline solid.

### Step 2: Synthesis of Heptamethoxy Red

1-bromo-2,4-dimethoxybenzene (CAS #17715-69-4) (4.23 g, 19.47 mmol) was added to a round bottom flask, and the flask flushed with nitrogen for 10 minutes. Anhydrous ether (CAS #60-29-7) (80 mL) was added, followed by the drop wise addition of n-butyllithium (CAS #109-72-8) in hexane (CAS #110-54-3) (1.6 M, 12.2 mL). The cloudy mixture was stirred at room temperature for 10 minutes. Methyl 2,4,6-trimethoxybenzoate (CAS #29723-28-2) (2.20 g, 9.74 mmol) was dissolved in ether (CAS #60-29-7), and added drop wise to the reaction mixture. After the addition was complete, the reaction was stirred for 3 minutes longer. The reaction was then poured into a separatory funnel containing 5% NH₄Cl (CAS #12125-02-9) (50 mL) and shaken until a color change was observed. The layers were separated, and the ether layer was dried over anhydrous Na₂SO₄ (CAS #7757-82-6), filtered, and rotovapped to dryness. The crude oil was placed in the freezer (6.02 g, 132% due to impurities).

### Example 2

### One Step Preparation of Heptamethoxy Red

Add (4.23 g, 19.47 mmol) 1-bromo-2,4-dimethoxybenzene (CAS #17715-69-4) to an appropriately sized round bottom flask. Attach a rubber septum to seal the flask.

Insert a needle into the septum as a vent and flush the round bottom flask with nitrogen for about 10 minutes.

Add (80 mL) anhydrous ether (CAS #60-29-7), followed by the drop wise addition of n-butyllithium (CAS #109-72-8) in hexane (CAS #110-54-3) (1.6 M, 12.2 mL).

Stir the cloudy mixture for 10 minutes and keep the round bottom flask on ice.

Dissolve (2.20 g, 9.74 mmol) of methyl 2,4,6-trimethoxybenzoate (CAS #29723-28-2) in about 20 ml of anhydrous ether (CAS #60-29-7) (more than ˜20 mL can be used if needed), and then add this drop wise to the reaction mixture.

After the addition is complete, stir the reaction mixture for about 3 minutes longer.

Pour the reaction mixture into a separatory funnel containing 5% NH₄Cl (aq) (CAS #12125-02-9) (50 mL) and shake until a color change is observed (pale orange).

The layers are allowed to separate, and dry the top ether layer with about 5 g anhydrous Na₂SO₄ (CAS #7757-82-6), filter, and rotovapped to dryness at 35-40° C. under 400 mbar.

Place the crude oil of heptamethoxy red (yellow-orange in color) into the freezer.

Yield is ˜3.1g.

### Example 3

### Preparation of Hexamethoxy Red in Gram Scale

Add (4.23 g, 19.47 mmol) 1-bromo-2,4-dimethoxybenzene (CAS #17715-69-4) to an appropriately sized round bottom flask.

Attach a rubber septum to seal the flask.

Insert a needle into the septum as a vent and flush the round bottom flask with nitrogen for about 10 minutes.

Add (80 mL) anhydrous ether (CAS #60-29-7), followed by the drop wise addition of n-butyllithium (CAS #109-72-8) in hexane (CAS #110-54-3) (1.6 M, 12.2 mL).

Stir the cloudy mixture for 10 minutes and keep the round bottom flask on ice.

Dissolve (2.20 g, 9.74 mmol) of methyl 2,4-dimethoxybenzoate (CAS #2150-41-6) in about 20 ml of anhydrous ether (CAS #60-29-7) (more than about 20 ml can be used if needed), and then add this drop wise to the reaction mixture.

After the addition is complete, stir the reaction mixture for about 3 minutes longer.

Pour the reaction mixture into a separatory funnel containing 5% NH₄Cl (aq) (CAS #12125-02-9) (50 mL) and shake until a color change is observed (pale orange).

The layers are allowed to separated, and dry the top ether layer with about 5 g anhydrous Na₂SO₄ (CAS #7757-82-6), filter, and rotovapped to dryness at 35-40° C. under 400 mbar.

Place the crude oil of hexamethoxy red (yellow-orange in color) into the freezer.

Yield is about 3.1 g.

### Example 4

### Preparation of Coating Solution

Prepare a 5% solution of ethyl cellulose in ethanol by adding 50 g of ethyl cellulose (CAS #9004-57-3) to 950 ml of 95% ethanol (CAS #64-17-5). Mix thoroughly.

Prepare a 1% Heptamethoxy Red solution in 5% ethyl cellulose by taking 990 ml of the above 5% ethyl cellulose in ethanol solution and add 10 ml of heptamethoxy red. Mix thoroughly.

The embodiments and examples described above are not intended to limit the invention. It should be understood that numerous modifications and variations are possible in accordance with the principles of this invention.

1. An adherent, non-acidic solution or homogeneous suspension which when dry is useful for determining the presence or absence of the growth by-products from contaminating microorganisms said solution or suspension comprises:
an adherent biocompatible polymer;
a biocompatible liquid; and
a plurality of indicator moieties which exhibit a first color or are colorless in the absence of bacterial growth by-products and a second color or are colorless in the presence of bacterial growth by-products.

2. The solution or homogeneous suspension of claim 1 wherein the adherent biocompatible polymer is a non-acidic polymer.

3. The solution or homogeneous suspension of claim 1 wherein the polymer is a non-transparent polymer.

4. The solution or homogeneous suspension of claim I wherein the polymer is a transparent polymer.

5. The solution or homogeneous suspension of claim 2 wherein the indicator moieties are pH indicator moieties uniformly dispersed throughout the solution or homogeneous suspension.

6. The solution or homogeneous suspension of claim 5 wherein the polymer is ethyl cellulose.

7. An adherent composition useful for determining the presence or absence of the growth by-products from contaminating microorganisms when said composition is adhered to a surface of a substrate and further wherein said composition comprises:
an adherent biocompatible polymer; and
a plurality of indicator moieties which exhibit a first color in the absence of bacterial growth by-products and a second color in the presence of bacterial growth by-products.

8. A substrate comprising on at least one surface thereof the adherent composition of claim 7.

9. The substrate of claim 8 wherein the composition is dissolved in a biocompatible liquid prior to adherence on a substrate.

10. The substrate of claim 9 wherein the composition adhered to said substrate is dried such that the biocompatible liquid content is less than 1 weight percentage.

11. (canceled)

12. The substrate of claim 8 wherein said substrate is human skin.

13. The substrate of claim 8 wherein said substrate is a medical device or component thereof and further wherein the surface comprising said composition is one which contacts bodily fluids.

14. (canceled)

15. The substrate of claim 8 wherein said substrate is a food or liquid storage device and further wherein the surface comprising said composition is one which contacts food or liquid.

16. The substrate of claim 15 which food or liquid storage device is selected from the group consisting of a sealable bag, a flexible wrap, a bulk container, and probes that interface with the interior contents of said container.

17-20. (canceled)

21. A non-acidic solution or homogeneous suspension which when dried is useful for determining the presence or absence of the growth by-products from contaminating microorganisms said solution or suspension comprises:
ethyl cellulose;
ethanol; and
a plurality of indicator moieties which exhibit no color in the absence of bacterial growth by-products and a color in the presence of bacterial growth by-products.

22-44. (canceled)

45. An applicator for dispensing the solution or homogeneous suspension of claim 1 comprising a storage chamber that holds the solution or homogeneous suspension and a dispensing mechanism.

46. The applicator of claim 45 wherein the dispensing mechanism is in the form of a sprayer, a brush, a flexographic or rotogravure printing press, or an elongated shaft with a sponge or non-flexible tip.

47. A. method for detecting the presence of a bacterial infection in a patient having a medical device or component thereof comprising a surface which is implanted or inserted into the patient such that at least a portion of the surface contacts bodily fluids of the patient which method comprises:
a) placing on at least a portion of the surface of the medical device that will be in contact with the bodily fluids of the patient the solution or homogeneous suspension of claim 1;
b) detecting the presence or absence of a colorimetric change in the composition, solution or suspension; and
c) correlating the presence or absence of a colorimetric change in the composition, solution or suspension to the presence of an active bacterial infection in the patient wherein a colorimetric change correlates to the presence of an active bacterial infection and the lack of a colorimetric change correlates to the absence of an active bacterial infection.

48-56. (canceled)
